(19) United States
(12) Patent Application Publication
(10) Pub. No.: US 2006/0269929 A1
(43) Pub. Date: Nov. 30, 2006

(54) Methods and kits for DNA purification on polymeric membranes at low ionic strength

(76) Inventors: Gerald E. Hall JR., Morrisville, PA (US); Barry E. Boyes, Wilmington, DE (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 11/139,933

(22) Filed: May 26, 2005

(51) Int. Cl. C12Q 1/68; C12P 19/34; C07H 21/04

(52) U.S. Cl. 435/6; 435/91.2; 536/25.4

(57) ABSTRACT

This invention relates to methods and kits for collecting DNA from nucleic acid capture materials such as polymeric membranes at low ionic strength. The methods and kits can be used to obtain DNA for use in subsequent procedures, such as amplification and labeling reactions while reducing the co-purification of contaminants.

### BACKGROUND

Commercially available DNA isolation systems often involve the use of anion-exchange chromatography. Anion-exchange chromatography is based on the interaction between negatively charged nucleic acid molecules and a positively charged support. DNA is typically bound under low salt conditions and eluted using a high salt buffer. Salt carryover can inhibit subsequent reactions in which the DNA is used, e.g., polymerase-based reactions such as necessary for amplification, labeling, sequencing and other types of processes. Silica-based methods typically require binding in high salt and elution in low salt. Carryover of salt and/or silica-based resins in elution steps can similarly produce undesirable contaminants that can interfere in the subsequent uses of isolated DNA.

### SUMMARY

The invention relates to methods and kits for DNA purification on polymeric membranes at low ionic strength.

In one embodiment, the invention relates to a method for collecting DNA from a sample. The method comprises contacting a polymeric DNA capture material with a sample comprising DNA, under conditions where DNA is captured by the polymeric DNA capture material, releasing DNA from the polymeric DNA capture material in a low ionic strength buffer and collecting released DNA.

In one aspect, the polymeric DNA capture material comprises polysulfone. In another aspect, the polymeric DNA capture material comprises polyvinylpyrrolidone (PVP). In still another aspect, the polymeric DNA capture material comprises polysulfone and polyvinylpyrrolidone (PVP).

In certain aspects, the polymeric DNA capture material comprises an asymmetric porous membrane comprising a first surface and a second surface wherein the first surface comprises pores having on average a larger diameter than the pores of the second surface. The size of the pores can vary, and in certain aspects, the diameter of the pores on the first surface ranges from about 0.1 μm to 100 μm while the diameter of pores on the second surface ranges from about 0.1-10 μm.

In certain aspects, the polymeric DNA capture material comprises a hydrophilic material. In one aspect, the polymeric DNA capture material comprises polysulfone. In another aspect, the polymeric DNA capture membrane comprises both polysulfone and polyvinylpyrrolidone (PVP). In still another aspect, the material is an asymmetric membrane. In one aspect, the material comprises a polysulfone/PVP asymmetric membrane. In another aspect, the material comprises a polysulfone asymmetric membrane. In still another aspect, the polymeric DNA capture membrane comprises PVDF.

In one embodiment, the low ionic strength buffer comprises less than about 0.5M of a salt. In one aspect, the low ionic strength buffer comprises less than about 0.25 M of a salt. In another aspect, the low ionic strength buffer comprises less than about 0.05M of a salt. In certain aspects, the salt is a chaotropic salt, which can include, but is not limited to, guanidine isothiocyanate, guanidine HCl, sodium perchlorate, ammonium thiocyanate, sodium iodide, or a combination thereof.

In a further aspect, the low ionic strength buffer comprises no salt. In another embodiment, the low ionic strength buffer comprises at least about 20% of a low molecular weight alcohol. In one aspect, the low ionic strength buffer comprises at least about 40% of a low molecular weight alcohol. In another aspect, the low ionic strength buffer comprises at least about 50% of a low molecular weight alcohol. In a further aspect, the low molecular weight alcohol comprises isopropanol.

The invention further relates to kits. In one aspect, a kit according to the invention comprises a polymeric DNA capture material and a DNA capture buffer comprising a low ionic strength buffer. In certain aspects, the polymeric DNA capture material comprises polysulfone, polyvinylpyrrolidone (PVP), or a combination of polysulfone and polyvinylpyrrolidone. In certain aspects, the DNA capture material comprises an asymmetric porous membrane comprising a first surface and a second surface wherein the first surface comprises pores having on average a larger diameter than the pores of the second surface. In one aspect, the pores on the first surface range in diameter from about 0.1 μm to 100 μm and the pores on the second surface range in diameter from about 0.1-10 μm. In another aspect, the polymeric DNA capture material comprises a hydrophilic material. In a further aspect, the polymeric DNA capture material comprises a polysulfone/PVP asymmetric membrane. In still a further aspect, the polymeric DNA capture material comprises a polysulfone asymmetric membrane.

In one embodiment, the low ionic strength buffer comprises less than about 0.5M salt. In one aspect, the DNA capture buffer comprises less than about 0.25 M salt. In another aspect, the DNA capture buffer comprises less than about 0.05M salt. In one aspect the salt is a chaotropic salt. Exemplary salts include, but are not limited to, guanidine isothiocyanate, guanidine HCl, sodium perchlorate, ammonium thiocyanate, or sodium iodide. However, in another embodiment, the low ionic strength buffer does not comprise salt.

In another embodiment, the DNA capture buffer comprises at least about 20% of a low molecular weight alcohol. In one aspect, the DNA capture buffer comprises at least about 40% of a low molecular weight alcohol. In another aspect, the DNA capture buffer comprises at least about 50% of a low molecular weight alcohol. The kit of claim 21, wherein the DNA capture buffer comprises at least about 50% of a low molecular weight alcohol. In certain aspects, the low molecular weight alcohol comprises ethanol, methanol, n-propanol or isopropanol.

The invention further relates to a method for collecting DNA from a sample that comprises contacting a polymeric DNA capture material with a sample having greater than about 10 μg, at least about 50 μg or at least about 100 μg of DNA, under conditions where DNA is captured by the polymeric material, releasing DNA from the polymeric DNA capture material, and collecting released DNA. In certain aspects, the DNA is less than about 500 base pairs, less than about 200 base pairs, less than about 100 base pairs or less than about 50 base pairs. In certain aspects, the DNA is obtained from a formalin-fixed sample, such as from a paraffin-embedded formalin fixed sample. In other aspects, the DNA has been altered or copied by a DNA modification or polymerization reaction (e.g., such as an amplification reaction) prior to contacting with the polymeric DNA capture material. In certain aspects, the DNA is labeled prior to contacting with the polymeric DNA capture material.

In still other aspects, DNA collected using methods according to the invention is contacted with a nucleic acid array. In certain aspects, the copy number of one or more DNA molecules in the sample is determined. In still other aspect, DNA molecules comprising recognition sites for selected DNA binding proteins are obtained from the sample and bound to the DNA capture material.

### BRIEF DESCRIPTION OF THE FIGURES

The objects and features of the invention can be better understood with reference to the following detailed description and accompanying drawings.

FIG. 1 is a graph showing the effect of ionic strength and alcohol concentration on the purification of DNA with a 0.8 μm polysulfone/PVP asymmetric membrane isolation column. Ten micrograms of restriction enzyme-digested calf thymus DNA was applied to a 0.8 μm polysulfone/PVP asymmetric membrane isolation column in buffers containing increasing concentrations of guanidine isothiocyanate and isopropanol concentrations. Data is given as a percentage of DNA recovered from spectrometry at 260 nm.

FIG. 2 is a graph providing a comparison of RNA and DNA purification on MMM membranes at various ionic strengths. Ten micrograms of restriction enzyme-digested calf thymus genomic DNA was applied to 0.8 μm polysulfone/PVP asymmetric membrane isolation columns in buffers containing increasing guanidine isothiocyanate concentrations. Data is given as a percentage of DNA recovered from spectrometry at 260 nm. While DNA binds at low ionic strength, RNA requires higher ionic strength for sufficient purification.

FIG. 3 is a graph providing a comparison of various solid supports and binding conditions for purification of DNA. Ten micrograms of restriction enzyme digested calf thymus genomic DNA was applied to various isolation columns (polysulfone/PVP asymmetric membrane, polysulfone asymmetric membrane, PVDF, glass fiber and a Qiagen QIAquick spin column) in various buffers: H₂O/50% isopropanol (“H₂O/IPA”); 1M guanidine isothiocyanate/50% isopropanol (“0.25×GITC/IPA”); 1 M guanidine isothiocyanate (“0.25×GITC); and Qiagen Qiaquick buffer PB, containing guanidine HCL and isopropanol at unknown concentrations). Data is given as μg of DNA recovered from spectrometry at 260 nm.

### DETAILED DESCRIPTION

The present invention pertains to methods and reagents used for collecting and/or isolating DNA. The methods can be used for preparing DNA for subsequent reactions such as amplification and labeling with minimal salt contamination from binding and elution steps. For example, the methods can be used for preparing targets for analysis of gene expression and genome-wide analysis of regulatory events (e.g., binding of DNA binding factors) and copy number. In one embodiment, the methods are used to prepare target nucleic acids for binding to arrays for performing gene expression analysis and a genome assay, e.g., such as comparative genomic hybridization, location analysis assay and the like.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates that may need to be independently confirmed.

It must be noted that as used herein and in the appended claims, the singular forms “a”, “an”, and “the” include plural referents unless the context clearly dictates otherwise.

“May” refers to optionally.

When two or more items (for example, elements or processes) are referenced by an alternative “or”, this indicates that either could be present separately or any combination of them could be present together except where the presence of one necessarily excludes the other or others.

The following definitions are provided for specific terms, which are used in the following written description.

The term “binding” refers to two molecules associating with each other to produce a stable composite structure under the conditions being evaluated (e.g., such as conditions suitable for RNA isolation). Such a stable composite structure may be referred to as a “binding complex”.

As used herein, the term “RNA” or “oligoribonucleotides” refers to a molecule having one or more ribonucleotides. The RNA can be single, double or multiple-stranded (e.g., comprise both single-stranded and double-stranded portions) and may comprise modified or unmodified nucleotides or non-nucleotides or various mixtures and combinations thereof.

As used herein, the term “DNA” or “deoxyribonucleotides” refers to a molecule comprising one or more deoxyribonucleotides. The DNA can be single, double or multiple-stranded (e.g., comprise both single-stranded and double-stranded portions) and may comprise modified or unmodified nucleotides or non-nucleotides or various mixtures and combinations thereof.

As used herein “complementary sequence” refers to a nucleic acid sequence that can form hydrogen bond(s) with another nucleic acid sequence by either traditional Watson-Crick or other non-traditional types (for example, Hoogsteen type) of base-paired interactions.

In certain embodiments, two complementary nucleic acids may be referred to as “specifically hybridizing” to one another. The terms “specifically hybridizing,” “hybridizing specifically to” and “specific hybridization” and “selectively hybridize to,” are used interchangeably and refer to the binding, duplexing, complexing or hybridizing of a nucleic acid molecule preferentially to a particular nucleotide sequence under stringent conditions. “Hybridizing” and “binding”, with respect to polynucleotides, are used interchangeably.

The term “reference” is used to refer to a known value or set of known values against which an observed value may be compared.

It will also be appreciated that throughout the present application, that words such as “cover”, “base” “front”, “back”, “top”, “upper”, and “lower” are used in a relative sense only.

As used herein, the term “contacting” means to bring or put together. As such, a first item is contacted with a second item when the two items are brought or put together, e.g., by touching them to each other.

As used herein, the term “solid phase” or “solid substrate” includes rigid and flexible solids. Examples of solid substrates include, but are not limited to, gels, fibers, microspheres, spheres, cubes, particles of other shapes, channels, microchannels, capillaries, walls of containers, membranes and filters.

As used herein, the term “silica-based” is used to describe SiO₂ compounds and related hydrated oxides and does not encompass silicon carbide compositions, which are described herein.

As used herein, a “nucleic acid-binding material,” stably binds a nucleic acid (e.g., such as double-stranded, single-stranded or partially double-stranded DNA, RNA or modified form thereof). By “stably binds” it is meant that under defined binding conditions the equilibrium substantially favors binding over release of the subcellular component, and if the solid substrate containing a selected bound subcellular component is washed with buffer lacking the component under these defined binding conditions, substantially all the component remains bound. In particular embodiments the binding is reversible. As used herein, the term “reversible” means that under defined elution conditions the bound subcellular component is predominantly released from the subcellular component-binding material and can be recovered (e.g., in solution). In particular embodiments, at least 50%, at least 60%, at least 90%, or at least 95% of the bound nucleic acid component is released under the defined elution conditions.

As used herein, a “nucleic acid capture material” is one which preferentially retains or traps or remains associated with nucleic acids to remove a nucleic acid from a solution. A nucleic acid capture material may, but does not necessarily, bind to a nucleic acid molecule.

“Washing conditions” include conditions under which unbound or undesired components are removed from a module of a device described below.

The term “assessing” “inspecting” and “evaluating” are used interchangeably to refer to any form of measurement, and includes determining if an element is present or not. The terms “determining,” “measuring,” “assessing,” and “assaying” are used interchangeably and include both quantitative and qualitative determinations. Assessing may be relative or absolute. “Assessing the presence of” includes determining the amount of something present, as well as determining whether it is present or absent.

A chemical “array”, unless a contrary intention appears, includes any one, two or three-dimensional arrangement of addressable regions bearing a particular chemical moiety or moieties (for example, biopolymers such as polynucleotide sequences) associated with that region. For example, each region may extend into a third dimension in the case where the substrate is porous while not having any substantial third dimension measurement (thickness) in the case where the substrate is non-porous. An array is “addressable” in that it has multiple regions (sometimes referenced as “features” or “spots” of the array) of different moieties (for example, different polynucleotide sequences) such that a region at a particular predetermined location (an “address”) on the array will detect a particular target or class of targets (although a feature may incidentally detect non-targets of that feature). Such a region may be referred to as a “feature region”. The target for which each feature is specific is, in representative embodiments, known. An array feature is generally homogenous in composition and concentration and the features may be separated by intervening spaces (although arrays without such separation can be fabricated).

Additional terms relating to arrays and the hybridization of nucleic acids to such arrays may be found, for example, in U.S. Pat. No. 6,399,394.

In one embodiment, the invention relates to the use of a device that comprises a module comprising a DNA capture material for separating DNA in a sample (e.g., such as genomic DNA, cloned DNA, DNA fragments (e.g., such as restriction digested or labeled DNA fragments, etc.) from other components. In one aspect, the other components comprise proteins, lipids, carbohydrates, and other non-DNA components of a biological sample. In another aspect, the other components comprise proteins, nucleotides, salts, and other non-DNA components of an in vitro DNA modification or polymerization reaction. In one aspect, the device comprises a housing having an open end and comprises walls defining a lumen that receives or comprises a module having DNA capture material, and comprises a closed bottom end. The module comprising the DNA capture material may be removable from the housing or an integral part of the housing or some combination thereof. As used herein, the term “module” refers to a functional element or unit in the device that may or may not be removable from the device.

The shape and dimensions of the housing may vary. However, in one embodiment, the housing is shaped like a tube or column. In another aspect, the housing is shaped like a tube and the module comprising the DNA capture material is provided in the form of a column that fits into the tube, the remaining space defining a collection compartment or chamber for receiving flow through or molecules eluted or otherwise released from the DNA capture material.

In certain aspects, a plurality of device housings is provided in a holder or container or rack and modules comprising DNA capture materials (e.g., columns) may be inserted into the lumen of each of the housings. In one aspect, the plurality of device housings is provided as a single unit (e.g., molded as a single unit from a plastic or other suitable material) comprising a plurality of lumens for receiving a plurality of columns.

Individual modules of the device may be separated from each other one at a time, e.g., by unscrewing or snapping apart. Likewise, the housing may be made from a variety of materials, including but not limiting to, a polymeric material such as plastic, polycarbonate, polyethylene, PTFE, polypropylene, polystyrene and the like.

The DNA capture material preferentially captures, e.g., retains, traps and/or binds DNA under DNA capture conditions. DNA can include genomic DNA, which may or may not have been previously crosslinked (e.g., to DNA binding proteins), fragmented, amplified and/or labeled. In certain aspects, DNA is reverse transcribed from RNA in the sample. In certain aspects, DNA molecules captured on the DNA capture material includes fragments of about 500 base pairs or less, about 200 base pairs or less, or about 100 base pairs or less. In certain aspects, the DNA capture material captures DNA at pHs which are less than pH 8.0, e.g., at about pH 6.5-7.5.

Further, in certain aspects, the module comprising the nucleic acid capture material does not comprise a matrix for anion exchange.

The form of the DNA capture material can vary. In one aspect, the DNA capture material comprises a fibrous, whisker, porous, or polymeric material or some combination thereof. For example, the DNA capture material can be provided in the form of fibers, whiskers, beads, membranes, filters, and the like.

In a particular aspect of this invention, the module comprising the DNA capture material comprises at least one layer of fiber filter material along with a retainer ring that is disposed adjacent to a first surface of the fiber filter material that securely retains the layer(s) of fiber filter material so that they do not excessively swell when sample is added. In one aspect, a frit is provided which is disposed adjacent to a second surface of the fiber filter material. The frit may assist in providing support so that the materials of the filter fibers do not deform. In one aspect, the frit is composed of polyethylene of about 90 μm thick.

In certain aspects, the DNA capture material comprises a porous filter. The pore size of the filter may be uniform or non-uniform. Where a plurality of filters are used, the pore size of each filter may be the same or different. In another aspect, suitable pore sizes may range from about 0.05 μm to about 10 μm.

In another embodiment, the module comprising the DNA capture material comprises one or more polymeric membranes, examples of which include, but are not limited to, polysulfone, polyvinylpyrrolidone (PVP) polysulfone, and composites thereof. In another aspect, the capture material comprises an asymmetric membrane with pores that gradually decrease in size from the upstream side to the downstream side. In one aspect, the membrane comprises pore sizes from about 0.1 μm to 100 μm. In another aspect, the membrane comprises pore sizes of from about 0.1 μm to 5 μm, or from about 0.1 μm to about 10 μm, or from 0.4 μm to 0.8 μm.

In still another aspect, the capture material comprises a hydrophilic material. An exemplary type of membrane is the polysulfone/PVP asymmetric filter (e.g., such as an MMM membrane, available from Pall Life Sciences).

In another embodiment, DNA capture material comprises polysulfone. In one aspect, the membrane comprises polysulfone and is an asymmetric porous membrane comprising pore sizes ranging from 0.02 to 10 μm or from about 0.02 to about 0.8 μm. In another aspect, the membrane is hydrophobic and/or hydrophilic. An example of such a membrane is a polysulfone asymmetric membrane (e.g., such as a BTS membrane, available from Pall Life Sciences).

Other exemplary DNA capture materials include, but are not limited to PVDF, nylon, nitrocellulose, and composites thereof.

In a further embodiment of the present invention, the module comprising DNA capture material comprises a column comprising an inlet and an outlet between which lies a chamber comprising a single or multiple layers of a polymeric membrane, as described above. A retainer ring and a frit can be disposed about the membrane(s) to retain them within the collection module. For example, a retainer ring may be disposed proximal to the inlet while a frit may be disposed proximal to the outlet. In one aspect, the membrane comprises a first surface and a second surface, the first surface having pores that are larger, on average, than the pores on the second surface. For example, in one aspect, the first surface has 30-40 μm diameter pores and the second surface has 0.1-10 μm diameter pores. In another aspect, the membrane comprises intermediate-sized pores between the first and second surface. In still another aspect, the larger diameter pores are on the upper side of the membrane while the smaller diameter pores (proximal to the collection module of the device) are on the lower surface.

In one embodiment, the invention further relates to methods of using the devices discussed above to isolate DNA. In one aspect, DNA capture on the polymeric membrane occurs at less than 500 mM salt, less than 100 mM salt, less than 10 mM salt, or no salt with greater than about 50% lower alcohol, greater than about 25% lower alcohol, greater than about 10% lower alcohol. DNA release from the DNA capture material occurs at low ionic strength in the absence of alcohol, e.g., in a solution comprising less than about 10 mM concentration of a salt, thereby minimizing the potential for interference by salt carryover in downstream applications In one embodiment, a sample is homogenized in an extraction buffer prior to contacting the sample with the module comprising the DNA capture material. Sample sources include, but are not limited to animals, plants, fungi (e.g., such as yeast), bacteria, and portions thereof. In one aspect, the animal can be a mammal, and in a further aspect, the mammal can be a human. Sample sources may additionally include virally infected cells, as well as transgenic animals and plants or otherwise genetically modified animals and plants. In addition, the sample can originate from experimental protocols, for example, from a collection of DNA crosslinked to DNA binding proteins where the crosslinking has been reversed (e.g., by heating), after fragmentation (e.g., in a DNase footprinting reaction or location analysis protocol, or after storage, for example, where the sample has been exposed to a formalin fixative, and extracted from paraffin), from an amplification reaction or from the products of an enzymatic reaction (e.g., a polymerization and/or transcription reaction), and/or from a labeling reaction. In certain aspects, as discussed above, the DNA contacted to the DNA capture material is about 500 base pairs or less, about 200 base pairs or less or about 100 base pairs or less. In certain aspects, the DNA is from a sample of cDNA molecules, vector molecules (e.g., phage DNA, plasmid DNA, viral DNA and the like), or other recombinantly engineered molecules and the sample does not necessarily comprise genomic DNA.

Amounts of DNA applied to the DNA capture material can vary, however, in certain aspects, greater than about 10 μg of DNA, greater than about 50 μg of DNA, greater than about 100 μg of DNA, greater than about 200 μg of DNA, or greater than about 300 μg of DNA can be applied to a DNA capture material of 35 mm², with greater than about 85% or greater recovery of DNA.

In certain aspects, the sample is applied to the DNA capture material in a solvent, for example, a low molecular weight alcohol such as isopropanol, ethanol, methanol, n-propanol. In one aspect, the sample is applied in at least about 10% isopropanol, at least about 20% isopropanol, at least about 30% isopropanol, at least about 40% isopropanol, or at least about 50% isopropanol or another low molecular weight alcohol. In one aspect, the sample is applied, in the absence of a salt, and the presence of at least about 20%, at least about 30%, at least about 40% or at least about 50% isopropanol or another low molecular weight alcohol. In another aspect, the sample is applied in 0.05M salt and at least about 40% or at least about 50% isopropanol or another low molecular weight alcohol. In still another aspect, the sample is applied in 0.1M salt and at least about 40% or at least about 50% isopropanol or another low molecular weight alcohol. In a further aspect, the sample is applied in at least about 50% isopropanol or another low molecular weight alcohol and less than 1M salt, less than 0.5M salt, less than about 0.25M salt, less than about 0.1M salt, less than about 0.05M salt.

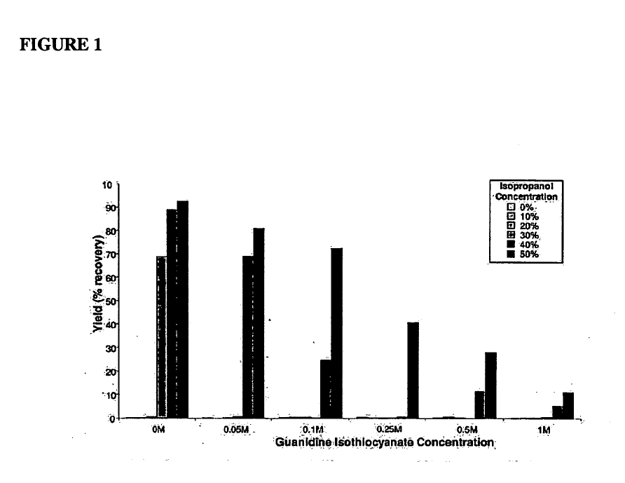

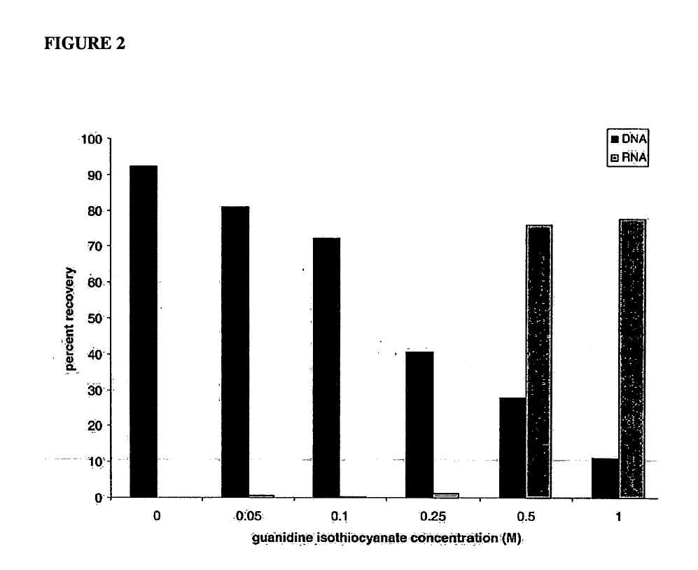

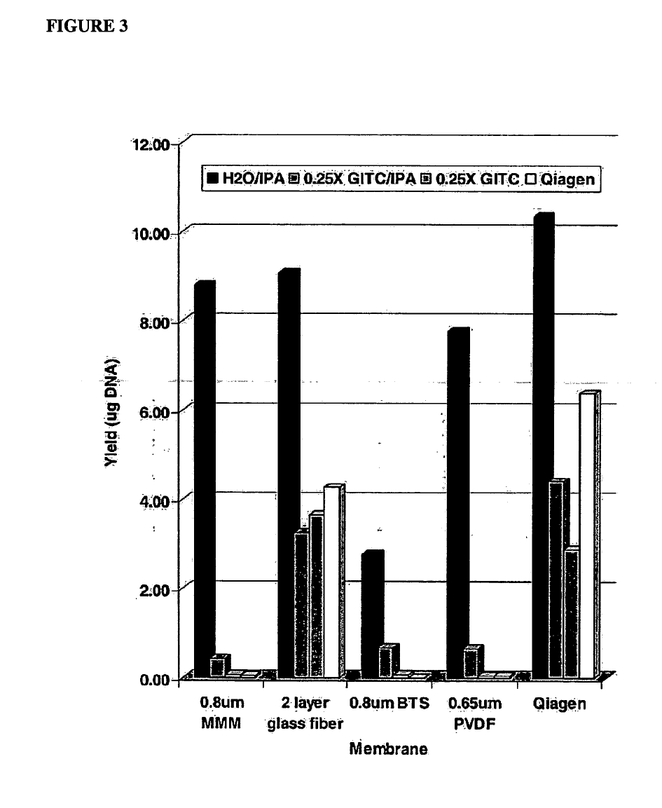

As shown in FIGS. 1 and 3, low ionic strength DNA capture buffers such as described above can be used to recover good yields of DNA from a sample while minimizing the potential for salt contamination in subsequent reactions. As shown in FIG. 2, low ionic strength buffer (e.g., less than about 0.5 M guanidine isothiocyanate) can be used to effectively capture DNA on a nucleic acid capture material, such as one comprising a polymer, while higher ionic strength buffers (about 0.5 M or greater guanidine isothiocyanate) are required to capture RNA in a sample. Thus, in low ionic strength buffer, DNA can be captured on a DNA capture material according to the invention while RNA flows through the material and can be separated away from DNA in the sample.

DNA can be released or eluted from the DNA capture material in a low ionic strength aqueous buffer such as water, 10 mM Tris, or 10 mMTris-1 mM EDTA, in the absence of a low molecular weight solvent.

The quality and/or quantity of nucleic acids collected may be evaluated and optimized using methods well known in the art, such as obtaining an A260/A280 ratio, evaluating an electrophoresed sample, or by using the Agilent Technologies® Bioanalyzer 2100 (part no. G2938B, Agilent Technologies®, Palo Alto, Calif.) as per manufacturer's instructions.

Collected DNA can be used in a variety of assays, such as comparative genomic hybridization, location analysis, and the like. In one aspect, collected DNA is used in an array CGH assay, as described in, WO2004058945, for example. In still another aspect, collected DNA is used in an array-based location analysis assay, such as described in U.S. Pat. 6,410,243, for example. In still other aspects, for example, DNA is labeled in a polymerization-based reaction (e.g., primer extension, nick translation, amplification, and the like), the DNA capture material can be used to separate labeled DNA from unincorporated labeled nucleotides. The collected labeled DNA can be subsequently used in an appropriate assay, such as for example in an array-based assay. Labeled DNA can be genomic DNA (included fragmented genomic DNA), amplified DNA, unamplified DNA, cDNA reverse transcribed from RNA, and the like.

In one embodiment, the invention further provides kits. In one aspect, a kit according to the invention provides a device comprising a module comprising a DNA capture material as described above and one or more collection modules for receiving released/eluted DNA. The kit may additionally include buffers suitable for collecting DNA and, optionally RNA from a sample. In one aspect, the kit comprises DNA capture buffer for facilitating capture of DNA molecules on the DNA capture material and/or a DNA releasing buffer for releasing/eluting DNA from the nucleic acid capture material.

In one aspect, the DNA capture buffer comprises a low ionic strength solution. For example, the DNA capture buffer can comprise less than about 1 M of a chaotropic salt, such as guanidine isothiocyanate. In certain aspects, the DNA capture buffer further comprises at least about 20% of an organic solvent, such as a low molecular weight alcohol. At higher solvent concentrations (e.g., 50-80%), salt concentration appears to have little effect on DNA capture and higher concentrations of salt can be used.

The DNA releasing buffer comprises a low ionic strength aqueous solution, e.g., less than about 100 mM salt, and in some aspects, may be water or TE.

In a further aspect, the kit comprises labeling reagents for labeling DNA, primers and suitable polymerases for incorporating labels into a DNA molecule, and the like.

In one aspect, the kit comprises reagents for performing a comparative genome hybridization (CGH) assay, or location analysis assay, e.g., such as reagents for performing a whole genome amplification reaction. Such reagents can include, but are not limited to: random primers, degenerate primers, primers that bind to universal adaptors or linker molecules, polymerases (e.g., such as phi29, the Klenow fragment of DNA pol I, etc), helicases, single-stranded binding proteins and the like. Reagents for performing location analysis can further include a crosslinking agent such as buffered formalin, formaldehyde, and the like. In still another aspect, the kit can comprise one or more arrays. Instructions for a practitioner to practice the invention may also included. Array CGH assays may be performed as described in WO2004058945, for example. Location analysis assays may be performed as described in U.S. Pat. No. 6,410,243, for example. Such array assays can be performed in parallel or sequentially with gene expression assays on the same or different arrays.

While this invention has been particularly shown and described with references to specific embodiments, it will be understood by those skilled in the art that various changes in form and details may be made therein without departing from the spirit and scope of the invention as defined by the appended claims.

References, patents, and patent applications cited herein are incorporated by reference in their entireties herein.

What is claimed is:

1. A method for collecting DNA from a sample comprising
contacting a polymeric DNA capture material with a sample comprising DNA, under conditions where DNA is captured by the polymeric DNA capture material;
releasing DNA from the polymeric DNA capture material and collecting released DNA.

2. The method of claim 1, wherein DNA is captured by the polymeric DNA capture material under low ionic strength conditions.

3. The method of claim 1, wherein DNA is released from the polymeric DNA capture material under low ionic strength conditions.

4. The method of claim 1, wherein the DNA comprises genomic DNA.

5. The method of claim 1, wherein the polymeric DNA capture material comprises polysulfone.

6. The method of claim 1, wherein the polymeric DNA capture material comprises polyvinylpyrrolidone (PVP).

7. The method of claim 5, wherein the polymeric DNA capture material comprises polysulfone and polyvinylpyrrolidone (PVP).

8. The method of claim 1, wherein the polymeric DNA capture material comprises an asymmetric porous membrane comprising a first surface and a second surface wherein the first surface comprises pores having on average a larger diameter than the pores of the second surface.

9. The method of claim 1, wherein the pores on the first surface range from about 0.1 μm to 100 μm and the pores on the second surface range from about 0.1-10 μm.

10. The method of claim 1, wherein the polymeric DNA capture material comprises a hydrophilic material.

11. The method of claim 1, wherein the low ionic strength buffer comprises less than about 0.5M salt.

12. The method of claim 1, wherein the low ionic strength buffer comprises less than about 0.25 M salt.

13. The method of claim 1, wherein the low ionic strength buffer comprises less than 0.05M salt.

14. The method of claim 1, wherein the low ionic strength buffer comprises a chaotropic salt.

15. The method of claim 11, wherein the salt comprises guanidine isothiocyanate, guanidine HCl, sodium perchlorate, ammonium thiocyanate, sodium iodide, or a combination thereof.

16. The method of claim 1, wherein the low ionic strength buffer comprises at least about 20% of a low molecular weight alcohol.

17. The method of claim 1, wherein the low ionic strength buffer comprises at least about 40% of a low molecular weight alcohol.

18. The method of claim 1, wherein the low ionic strength buffer comprises at least about 50% of a low molecular weight alcohol.

19. The method of claim 16, wherein the low molecular weight alcohol comprises ethanol, methanol, n-propanol or isopropanol.

20. A kit comprising a polymeric DNA capture material and a DNA capture buffer comprising a low ionic strength buffer

21. The kit of claim 20, wherein the polymeric DNA capture material comprises polysulfone.

22. The kit of claim 20, wherein the polymeric DNA capture material comprises polyvinylpyrrolidone.

23. The kit of claim 21, wherein the polymeric DNA capture material comprises polysulfone and polyvinylpyrrolidone (PVP).

24. The kit of claim 20, wherein the polymeric DNA capture material comprises an asymmetric porous membrane comprising a first surface and a second surface wherein the first surface comprises pores having on average a larger diameter than the pores of the second surface.

25. The kit of claim 20, wherein the pores on the first surface range from about 0.1 μm to 100 μm and the pores on the second surface range from about 0.1-10 μm.

26. The kit of claim 20, wherein the polymeric DNA capture material comprises a hydrophilic material.

27. The kit of claim 20, wherein the low ionic strength buffer comprises less than about 0.5M salt.

28. The kit of claim 20, wherein the DNA capture buffer comprises less than about 0.25 M salt.

29. The kit of claim 20, wherein the DNA capture buffer comprises less than about 0.05M salt.

30. The kit of claim 20, wherein the DNA capture buffer comprises at least about 20% of a low molecular weight alcohol.

31. The kit of claim 20, wherein the DNA capture buffer comprises at least about 40% of a low molecular weight alcohol.

32. The kit of claim 20, wherein the DNA capture buffer comprises at least about 50% of a low molecular weight alcohol.

33. The kit of claim 27, wherein the salt comprises a chaotropic salt.

34. The kit of claim 33, wherein the chaotropic salt comprises guanidine isothiocyanate, guanidine HCl, sodium perchlorate, ammonium thiocyanate, sodium iodide, or a combination thereof.

35. The kit of claim 30, wherein the low molecular weight alcohol comprises ethanol, methanol, n-propanol or isopropanol.

36. A method for collecting DNA from a sample comprising
contacting a polymeric DNA capture material with a sample comprising greater than about 10 μg of DNA, under conditions where DNA is captured by the polymeric material;
releasing DNA from the polymeric DNA capture material; and
collecting released DNA.

37. The method of claim 36, wherein the method comprises contacting the DNA capture material with a sample comprising at least about 50 μg of DNA.

38. The method of claim 37, wherein the method comprises contacting the DNA capture material with a sample comprising at least about 100 μg of DNA.

39. The method of claim 1, wherein the DNA is less than about 500 base pairs.

40. The method of claim 1, wherein the DNA is less than about 200 base pairs.

41. The method of claim 1, wherein the DNA is less than about 100 base pairs.

42. The method of claim 1, wherein the DNA is obtained from a formalin-fixed sample.

43. The method of claim 1, wherein the DNA has been altered or copied by a DNA modification or polymerization reaction prior to contacting with the polymeric DNA capture material.

44. The method of claim 1, wherein the DNA is labeled prior to contacting with the polymeric DNA capture material.

45. The method of claim 1, wherein the DNA is amplified prior to contacting with the polymeric DNA capture material.

46. The method of claim 1, wherein the collected DNA is contacted with a nucleic acid array.

47. The method of claim 46, wherein the copy number of one or more DNA molecules in the sample is determined.

48. The method of claim 1, wherein DNA molecules comprising recognition sites for selected DNA binding proteins are obtained from the sample and bound to the DNA capture material.

49. The method of claim 48, wherein collected DNA molecules comprising the recognition sites for selected DNA binding proteins are contacted to a nucleic acid array.

50. The method of claim 2, wherein the low ionic strength conditions includes an absence of salt.

51. The kit of claim 27, wherein the low ionic strength buffer does not comprise salt.
